(19) United States
(12) Patent Application Publication
(10) Pub. No.: US 2017/0010249 A1
(43) Pub. Date: Jan. 12, 2017

(54) METHOD FOR DETECTING CADMIUM AND KIT FOR DETECTING CADMIUM USING FLUORESCENCE INTENSITY ACCORDING TO FORMATION OF CADMIUM NANOCLUSTER

(71) Applicant: GWANGJU INSTITUTE OF SCIENCE AND TECHNOLOGY, Gwangju (KR)

(72) Inventors: Min-Gon KIM, Gwangju (KR); Hyo-Young MUN, Gwangju (KR); Yu-Seon NO, Gwangju (KR); Eun-Jung JO, Gwangju (KR)

(21) Appl. No.: 15/207,165

(22) Filed: Jul. 11, 2016

(30) Foreign Application Priority Data
Jul. 10, 2015 (KR) 10-2015-0098667

(51) Int. Cl. G01N 33/20; G01N 21/64; G01N 33/84

(52) U.S. Cl. CPC G01N 33/20; G01N 33/84; G01N 21/6428; G01N 2333/765; G01N 2201/061

(57) ABSTRACT

The present invention relates to a method for detecting cadmium capable of easily, rapidly, and simply detecting cadmium quantitatively and qualitatively by sequentially adding albumin and a reducing agent to a sample, the albumin and the reducing agent inducing synthesis of a cadmium nanocluster capable of measuring a change in fluorescence intensity. In addition, the albumin and the reducing agent used for the method for detecting cadmium have excellent sensitivity even though they are low-cost, such that concentration of the cadmium present in the sample is capable of being directly measured.

### BACKGROUND

1. Technical Field

The present invention relates to a method for detecting cadmium capable of easily detecting the cadmium quantitatively and qualitatively from various samples by combining cadmium ions with albumin and a reducing agent in high sensitivity to form a cadmium nanocluster, and a kit for detecting cadmium.

2. Description of the Related Art

In general, cadmium is accumulated in various organs such as a liver, kidneys, lungs in our bodies as we are exposed to environment such as water, soil or air, which causes serious diseases such as bone fractures, hypertension, renal dysfunction, cancer, etc. For this reason, the permissible level of cadmium is limited to about 3 ppb to 5 ppb in World Health Organization (WHO) and U.S. Environmental Protection Agency (U.S. EPA). The cadmium causes serious diseases even with a low concentration, and thus it is important to detect the cadmium for healthy human life.

Therefore, it is very important to develop a method for quantitatively detecting cadmium, which can be applied environmentally and biologically. So far, a method for detecting metal ions using analytical reagents having selectivity has been firstly used, and then, a number of researches into inventions and synthesis of a photochemical chemical sensor that detects specific metal ions have been developed. In addition to these methods, there are a cold vapour atomic absorption spectrometry by flame and electric heat, UV-visible spectrophotometry, X-ray absorption spectroscopy, and inductively coupled plasma emission spectroscopy, etc. These equipment-chemical methods have disadvantages in that cost is high due to high-priced equipments, and processes are complicated, a considerable time is consumed, such that it is not easy to utilize these methods unless users are an expert.

As an alternative to solve the disadvantages, it is demanded to develop a method for detecting cadmium having high specificity, a low cost for measurement, and easy handling, and being movable. As an example, a method for detecting cadmium using electrochemistry, quantity of electricity, potential difference, fluorescence, colorimetric method, etc., was invented. According to this method, the detection is capable of being relatively rapidly, simply, and inexpensively performed, but the measurement is difficult to be performed in a state having low sensitivity and being homogeneous, and accordingly, additional process such as washing is required. Therefore, there are still limitations in that processes are complicated, and a large amount of time is consumed.

### SUMMARY

It is an aspect of the present invention to provide a method for detecting cadmium capable of easily, rapidly, and inexpensively detecting cadmium with excellent sensitivity by using a detection composition including albumin and a reducing agent instead of various existing methods for detecting cadmium in which the cost is high and a large amount of time is consumed.

To this end, the present inventors found that when the low-cost albumin and the reducing agent are included in the detection composition, the albumin reacts with the cadmium to synthesize a cadmium nanocluster having fluorescence property, such that the cadmium in a sample is capable of being quantitatively and qualitatively detected by using the fluorescence property of the synthesized cadmium nanocluster, and completed the present invention.

In accordance with one aspect of the present invention, there is provided a method for detecting cadmium including: I) mixing and reacting a detection composition including albumin and a reducing agent with a sample containing the cadmium to obtain a cadmium nanocluster; and II) irradiating the cadmium nanocluster with ultraviolet rays, and obtaining a fluorescence spectrum.

In accordance with another aspect of the present invention, there is provided a composition for detecting cadmium including albumin and a reducing agent.

In accordance with another aspect of the present invention, there is provided a kit for detecting cadmium including: the composition for detecting cadmium as described above.

### BRIEF DESCRIPTION OF THE DRAWINGS

FIG. 1 is a schematic view illustrating a general process for detecting cadmium using a detection composition according to the present invention, and specifically, FIG. 1A illustrates a process of mixing the detection composition according to the present invention with a sample without containing cadmium, and FIG. 1B illustrates a process of mixing the detection composition according to the present invention with a sample containing cadmium.

FIG. 2 is a chart illustrating analysis results of fluorescence intensities derived from respective fluorescence images obtained by mixing detection compositions with samples according to Examples 1 to 6.

FIG. 3 is a chart illustrating analysis results of fluorescence intensities derived from respective fluorescence images obtained by mixing detection compositions with samples according to Examples 7 to 12.

FIG. 4 is a chart illustrating analysis results of fluorescence intensities derived from respective fluorescence images obtained by mixing detection compositions with samples according to Comparative Examples 1 to 6.

FIG. 5 is to confirm whether cadmium is detected by using human serum albumin and a reducing agent to samples for each concentration of cadmium ions, and is a graph illustrating analysis results of fluorescence intensities derived from respective fluorescence images obtained by mixing the detection compositions of Examples 13 to 20 with the samples under conditions of 12 h, 37° C., and 1 M NaOH before a detection time, a temperature, and concentration of reducing agent are optimized. An interposed image illustrates fluorescence intensities derived by adding human serum albumin and a reducing agent to samples for each concentration of cadmium ions.

FIG. 6 is a chart illustrating analysis results of fluorescence intensities derived from respective fluorescence images obtained by mixing the detection composition with the sample according to Example 6 at various temperatures.

FIG. 7 is a chart illustrating analysis results of fluorescence intensities derived from respective fluorescence images obtained by mixing the detection compositions with samples according to Examples 21 to 27.

FIG. 8 is a graph illustrating comparison in temperature between 55° C. and 37° C., wherein the detection is performed under all optimized detection conditions except for changing the temperature among detection conditions of the present invention.

FIG. 9 is a graph obtained by detecting cadmium from samples including various concentrations of cadmium ions under all optimized detection conditions. An interposed image illustrates fluorescence intensities derived by adding human serum albumin and a reducing agent to samples for each concentration of cadmium ions.

(A) in FIG. 10 is a Table illustrating analysis results of energy-dispersive X-ray spectroscopy (JEM-2100F manufactured by JEOL, Japan) between a detection solution of which a reaction is terminated and a Control Group obtained through a detection process of Test Example 8 according to the present invention using a sample in which cadmium ions are present and a sample in which cadmium ions are not present.

(B) in FIG. 10 is a Table illustrating an image of transmission electron microscopy (JEM-2100F manufactured by JEOL, Japan) of the detection solution of which the reaction is terminated obtained through the detection process of Test Example 8 according to the present invention using the sample in which cadmium ions are present.

(C) in FIG. 10 is a graph illustrating measurement results of dynamic light scattering (ELS-Z manufactured by Otsuka electronic, Japan) on the detection solution of which the reaction is terminated obtained through the detection process of Test Example 8 according to the present invention using the sample in which cadmium ions are present. (D) in FIG. 10 is a graph illustrating measurement results of fluorescence emission spectra (TECAN manufactured by Mannedorf, Switzerland) on the detection solution of which the reaction is terminated obtained through the detection process of Test Example 8 according to the present invention using the sample in which cadmium ions are present.

FIG. 11 is a graph illustrating high performance liquid chromatography (HPLC) analysis results on the detection solution of which the reaction is terminated obtained in the same manner as the detection process of Test Example 8 except for using samples having different concentrations of cadmium ions in order to confirm whether the cadmium nanocluster formed in the detection solution of which the reaction is terminated obtained by the detection process of Test Example 8 is affected depending on the concentration of cadmium ions.

FIG. 12 is a graph illustrating measurement results of MALDI-TOF MS (ASTA, KOREA) on the detection solution of which the reaction is terminated obtained in the same manner as the detection process of Test Example 8 except for using samples having different concentrations of cadmium ions in order to confirm whether the cadmium nanocluster formed in the detection solution of which the reaction is terminated obtained by the detection process of Test Example 8 is affected depending on the concentration of cadmium ions.

FIG. 13 illustrates fluorescence images obtained by performing the method for detecting cadmium of the present invention on real samples such as tap water (A), fountain water (B), and pond water (C) under optimized conditions, and analysis results of fluorescence intensities thereof, wherein the fluorescence images and the fluorescence intensities of all graphs except for (D) in FIG. 10 were obtained by using a ChemiDoc system (Bio-rad).

FIG. 14 is a graph illustrating analysis results of fluorescence intensities obtained by performing the method for detecting cadmium using human serum albumin and reducing agent according to the present invention on various samples including metal ions in order to confirm specificity of the method for detecting cadmium of the present invention to the cadmium ions. A in FIG. 14 is a graph illustrating analysis results of fluorescence images obtained by using ChemiDoc system (Bio-rad), and FIG. 14B is a graph illustrating fluorescence spectra obtained by using TECAN on gold ion (Au³⁺), chromium ion (Cr³⁺), and copper ion (Cu²⁺) having fluorescence intensity weaker than that of the cadmium ion (Cd²⁺) but exhibiting some degree of fluorescence.

FIG. 15 is a schematic diagram illustrating an exemplary embodiment of a kit for detecting cadmium according to the present invention.

FIG. 16 is a schematic diagram illustrating another exemplary embodiment of the kit for detecting cadmium according to the present invention.

### DETAILED DESCRIPTION

Hereinafter, various aspects and exemplary embodiments of the present invention will be described in detail.

The present inventors invented a method for detecting cadmium which is selective to high sensitivity of cadmium ions, and is easily, rapidly, and inexpensively performed by forming a cadmium nanocluster through a reaction of albumin and the cadmium and using a property in which the cadmium nanocluter exhibits fluorescence.

In general, a metal nanocluster refers to metal atoms that are formed and agglomerated in a size of several nanometers. Optical, catalytic, and magnetic measurement methods may be used depending on the size of cluster. As a method for forming the metal nanocluster, there are a reverse micelle synthesis method, a method for forming a cluster stabilized by a ligand using reduction of metal salts in a polar organic solvent, a method for decomposing a thermally unstable metal organic precursor and synthesizing the metal nanocluster in a non-aqueous solution, etc. These methods are toxic, expensive, and not favorable in environment.

On the contrary, the method for detecting cadmium according to the present invention is a detection method using albumin and a reducing agent, specifically, a novel method for detecting cadmium using formation of the cadmium nanocluster by reaction of the cadmium and albumin present in a sample, wherein the method is simple and cheap, and time for detection is rapid, as compared to the above-described methods in the related art.

In addition, after a detection composition including the sample, the albumin, and the reducing agent is mixed and time for detection is consumed, the method for detecting cadmium of the present invention is possible to detect and analyze cadmium as it is without additional separation and washing processes.

An aspect of the present invention is to provide a method for detecting cadmium including the following steps:

I) mixing and reacting a detection composition including albumin and a reducing agent with a sample containing the cadmium to obtain a cadmium nanocluster; and

II) irradiating the cadmium nanocluster with ultraviolet rays, and obtaining a fluorescence spectrum.

First, I) the detection composition including albumin and a reducing agent is mixed and reacted with the sample containing the cadmium to obtain the cadmium nanocluster.

Here, the reducing agent and the albumin may be simultaneously mixed in a sample solution, or the reducing agent may be mixed in an about 1 to 10 minutes after the albumin is primarily mixed in the sample solution. Both of a case in which the albumin and the reducing agent are simultaneously mixed in the sample solution and a case in which the reducing agent is mixed after the albumin is primarily mixed in the sample solution do not have a significant effect on synthesis and detection of the cadmium nanocluster, such that it is preferable to suitably select the cases depending on circumstances.

The albumin may be ovalbumin or human serum albumin or mixtures thereof. When bovine serum albumin, etc., excepting these albumins, are used, it is not reacted with the cadmium ions to form the cadmium nanocluster, such that it is not possible to detect whether the cadmium ions are present and concentration of cadmium ions.

In addition, the albumin is the most preferably the human serum albumin. The reason is that when the ovalbumin is used, the cadmium cluster may be formed through combination of ovalbumin and the cadmium, but the detection time is consumed for 12 hours, and in the detection time of 12 hours or less, the detection is not easy due to deteriorated sensitivity to the cadmium ions. Therefore, the human serum albumin is the most preferably used to detect the cadmium within a short period of detection time.

On the contrary, since the bovine serum albumin is not capable of forming the cadmium nanocluster at all within 12 hours or more of detection time, the bovine serum albumin is not useful for detecting cadmium at all.

The reducing agent is not specifically limited, but preferably, may be at least any one selected from the group consisting of sodium hydroxide (NaOH), ascorbic acid, formic acid, sodium borohydride (NaBH₄) and oxalic acid, and the most preferably, may be sodium hydroxide.

When the albumin meets the cadmium ions, the albumin surrounds the cadmium ions to sequester the cadmium ion, such that the cadmium ions are not enabled to be escaped, but are entrapped to form the cadmium cluster. During this process, when a reducing agent having a high pH is added, a reducing ability of albumin is activated, and the cadmium ions entrapped inside are directly reduced in-situ to form the cadmium nanocluster.

That is, when the sodium hydroxide having the highest pH among the reducing agents is used, as compared to the case in which other reducing agent is used, the ability of the albumin may be activated, the reduction of the cadmium ions entrapped in the albumin may be promoted, thereby rapidly forming the cadmium nanocluster formed of cadmium atoms, such that the detection time may be remarkably decreased to 2 hours.

Meanwhile, a concentration of the reducing agent in step I) is preferably 0.25 M 0.9 M. The reason is that when the concentration of the reducing agent is less than 0.25 M or more than 0.9 M, there is a problem in that time for preparing the cadmium nanocluster formed by reaction of the cadmium and the albumin is increased, such that time required for precise detection is increased. When the measurement is constrainedly performed under the condition in which the concentration of the reducing agent is less than 0.25 M or more than 0.9 M in a short period of detection time, there is a problem in that detection sensitivity is low.

In addition, when the detection composition including the albumin and the reducing agent is mixed with the sample containing the cadmium, the cadmium ions are reacted with the albumin to form the cadmium nanocluster exhibiting fluorescence intensity, wherein a diameter of the cadmium nanocluster vary depending on the concentration of the cadmium ions present in the sample solution. Specifically, as the concentration of the cadmium ions present in the sample solution is increased, the diameter of the cadmium nanocluster is increased, and as the concentration of the cadmium ions present in the sample solution is decreased, the diameter of the cadmium nanocluster is decreased.

As an example, 10 mM CdCl₂.2H₂O sample (500 μl) was reacted with the albumin, and as a result, the cadmium nanocluster having a diameter of about 4 to 5.5 nm was formed. One cadmium nanocluster includes about 20 of cadmium atoms.

The fluorescence intensity varies depending on an amount and a diameter of the formed cadmium nanocluster, and accordingly, the cadmium ions are capable of being quantitatively detected and qualitatively detected.

Meanwhile, the reaction of step I) needs to be performed at 50° C. to 60° C. to form the cadmium cluster. If the reaction is performed at a temperature condition less than 50° C., a reaction rate of the albumin and the cadmium ions is slow, such that a considerable time is consumed to form the cadmium nanocluster.

Further, when the measurement is constrainedly performed in a short period of detection time, it is performed in a state in which the reaction of the albumin and the cadmium ions is not completely achieved, such that there is a problem in that sensitivity to the cadmium ions is remarkably deteriorated.

In addition, when the reaction of step I) is performed at a temperature more than 60° C., protein modification is caused, which deteriorates an activity of the albumin, such that the albumin is not completely reacted with the cadmium ions, and eventually, the cadmium ions may not be detected.

In conclusion, in order to form the cadmium nanocluster to be capable of being detected by reacting the albumin and the cadmium ions present in the sample in step I), the cadmium ions may be detected in step I) with high sensitivity within a short period of detection time of 2 to 4 hours only in a range in which the above described concentration condition and the temperature condition of the reducing agent. However, when any one condition is out of the above conditions, there are problems in that the detection time required for detecting the cadmium is increased by 4 times or more, and the detection sensitivity is remarkably deteriorated.

The cadmium nanocluster has a property in which fluorescence is exhibited after ultraviolet irradiation. Finally, II) the cadmium nanocluster is irradiated with ultraviolet rays, and a fluorescence spectrum is obtained, and analyzed, thereby quantitatively and qualitatively detecting the cadmium ions.

The cadmium nanocluster is irradiated with ultraviolet rays, wherein the ray preferably has a wavelength between 300 to 400 nm, and more preferably, between 350 to 370 nm.

In addition, the method may further include deriving concentration of the cadmium ions on the basis of the fluorescence spectrum. Specifically, the detecting of the cadmium ions is characterized by confirming the presence and the concentration of the cadmium ions by measuring an increase in fluorescence intensity at a wavelength of 430 to 490 nm at the time of measuring the fluorescence spectrum.

Specifically, in order to confirm the presence of the cadmium ions, the fluorescence spectrum is obtained through the above-described process using a plurality of measurement samples obtained by diluting the cadmium ions which are detection targets to different scales, and the fluorescence intensity derived therefrom is used an element to obtain a line (calibration line) generated by an interpolation method or an extrapolation method is obtained on the basis of at least two measurement points. The concentration of the cadmium ions actually present in the sample may be approximately derived from the fluorescence intensity actually measured from the sample by using the calibration line.

In addition, the concentration of the cadmium ions actually present in the sample may be indirectly derived by calculating a slope from the calibration curve, and substituting the fluorescence intensity actually measured from the sample according to the detecting method of the present invention in the slope.

In the method for detecting cadmium according to the present invention, the concentration of the cadmium in the real sample may be accurately measured within a short period of time by directly mixing the detection composition with the sample without separately performing additional processes such as a washing step. In particular, it is possible to detect uniform cadmium ions without the washing process.

In the method for detecting cadmium according to the present invention, tolerance of the cadmium that is detectable is about 3 to 5 ppb, and a detection performance is 0.01 to 0.05 ppb.

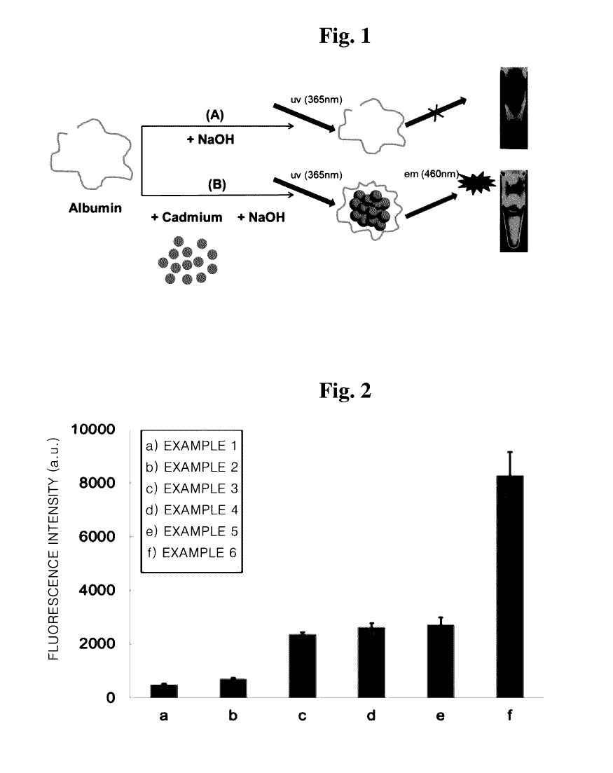

Overall processes of the above-described detection method are illustrated in FIG. 1. Referring to FIG. 1, when the detection solution including the albumin is added to the sample solution (A) without containing cadmium, the cadmium cluster by the cadmium is not formed, such that the fluorescence intensity is not exhibited. However, when the albumin and the reducing agent are mixed with the sample solution (B) containing the cadmium, respectively, wherein when the reducing agent is added thereto, the cadmium nanocluster is formed by the reaction of the cadmium and the albumin, and accordingly, it may be confirmed that the fluorescence intensity is exhibited after light irradiation.

The analysis of the fluorescence spectrum may be performed by analyzing fluorescence intensity at a wavelength in which the maximum fluorescence intensity is exhibited in the fluorescence spectrum of the cadmium nanocluster.

In the present invention, the cadmium nanocluster exhibits the strongest fluorescence signal when an absorption wavelength is 350 to 370 nm, approximately, 365 nm, and an emission wavelength is 400 to 500 nm, approximately, 460 nm ((D) in FIG. 10). The method for detecting cadmium from the cadmium nanocluster having the fluorescence property by utilizing this phenomenon is provided in the present invention.

As described above, in the method for detecting cadmium, the cadmium nanocluster is formed by sequentially or simultaneously adding the albumin and the reducing agent to the sample to react with the cadmium present in the sample.

The number of cadmium atoms used for forming one cluster varies depending on the concentration of the cadmium present in the sample, and accordingly, the cadmium nanocluster formed by the above-described process has a difference in fluorescence intensity generated depending on the concentration of the cadmium.

Therefore, whether the cadmium is present in the sample and the concentration of the cadmium may be measured by the difference in fluorescence intensity. Specifically, according to the detection method of the present invention, the cadmium nanocluster may be formed from the sample in which the cadmium ions are present, and the concentration of the cadmium present in the sample may be indirectly measured from the fluorescence intensity of the cadmium nanocluster.

In addition, another aspect of the present invention is to provide a composition for detecting cadmium including albumin and a reducing agent.

An amount of the albumin may be 20 to 30 parts by weight on the basis of 1 part by weight of the reducing agent. When the amount thereof is less than 20 parts by weight or more than 30 parts by weight on the basis of 1 part by weight of the reducing agent, there are problems in that the fluorescence intensity is 5000 a.u or less, that is, a limit of detection is low, and time required for the detection is significantly long.

The composition for detecting cadmium is used to detect whether the cadmium is present and the concentration of the cadmium from the sample, and the cadmium nanocluster is formed by reacting the cadmium present in the sample with the albumin, and measuring and analyzing the fluorescence intensity thereof, such that it may detect the whether the cadmium is present in the sample and the concentration of the cadmium.

In addition, the composition for detecting cadmium according to the present invention is not reacted with other materials present in the sample, but is specifically reacted only with the cadmium ions to form the cadmium nanocluster, such that only the cadmium may be easily, inexpensively, rapidly, and specifically detected with high sensitivity.

The albumin may be ovalbumin or human serum albumin or mixtures thereof. When bovine serum albumin, etc., excepting these albumins, are used, it is not reacted with the cadmium ions to form the cadmium nanocluster, such that it is not possible to detect whether the cadmium ions are present and concentration of cadmium ions.

In addition, the albumin is the most preferably the human serum albumin. The reason is that when the ovalbumin is used, the cadmium cluster may be formed through combination of ovalbumin and the cadmium, but the detection time is consumed for 12 hours, and in the detection time of 12 hours or less, the detection is not easy due to deteriorated sensitivity to the cadmium ions. Therefore, the human serum albumin is the most preferably used to detect the cadmium within a short period of detection time.

On the contrary, since the bovine serum albumin is not capable of forming the cadmium nanocluster at all within 12 hours or more of detection time, the bovine serum albumin is not useful for detecting cadmium at all.

The albumin may be in a form of liquid phase in which it is dissolved in various aqueous solutions such as distilled water, buffer solution, etc., or a form of solid powder, and the most preferably, the form of solid powder. The reason is that the solid powder form is capable of being stored for a long period of time, and being easily stored or transported.

When the albumin is in the liquid phase, even though general distilled water is used without using the buffer solution, the albumin is not easily denatured, and maintains high sensitivity to cadmium, such that any solution is usable without specific limitation as long as the solution does not denature the albumin.

The reducing agent may be at least any one selected from the group consisting of sodium hydroxide (NaOH), ascorbic acid, formic acid, sodium borohydride (NaBH₄) and oxalic acid.

The reducing agent may be in a form of liquid phase in which the reducing agent is dissolved in various aqueous solutions such as distilled water, buffer solution, etc., or a form of solid powder, preferably, the form of solid powder, and more preferably, solid-state NaOH.

When the reducing agent is in a liquid phase in which it is dissolved in various aqueous solution, any solution is usable without specific limitation as long as the solution does not denature the reducing agent.

When the albumin meets the cadmium ions, the albumin surrounds the cadmium ions to sequester the cadmium ion, such that the cadmium ions are not enabled to be escaped, but are entrapped to form the cadmium cluster. During this process, when a reducing agent having a high pH is added, a reducing ability of albumin is activated, and the cadmium ions entrapped inside are directly reduced in-situ to form the cadmium nanocluster.

That is, when the sodium hydroxide having the highest pH among the reducing agents is used, as compared to the case in which other reducing agent is used, the ability of the albumin may be activated, the reduction of the cadmium ions entrapped in the albumin may be promoted, thereby rapidly forming the cadmium nanocluster formed of cadmium atoms, such that the detection time may be remarkably decreased to 2 hours.

Most preferably, both of the albumin and the reducing agent are in the solid powder form. In order to be actually used in various places and environment, easy storage for a long period of time, easy transportation, light weight and small volume are required. However, when they are present in the liquid phase, the albumin may be denatured and may be sensitive to a change in external environmental conditions when stored for a long period of time, such that it is difficult to choose storage places. Further, additional instruments are required for adding the materials in accurate amounts or taking the solution out, and in addition, the additional instruments are required to be washed for every process to be detected, and accordingly, there is a limitation in places for actually using the kit. Further, when the albumin and the reducing agent are stored in a mixed form to reduce volume, there is a problem in that a protein structure of albumin is untangled to be inactivated.

Accordingly, it is preferred to have the albumin and the reducing agent preferably in the powder form, because there are advantages in that they are easily stored since they are not sensitive to the change in external environment, and a long period storage is possible since the protein structure of albumin is capable of being maintained for a long period of time. In addition, it is possible to directly detect cadmium from samples to be detected anywhere regardless of places at which the samples are positioned since the confirmation of cadmium is directly performed by adding the albumin and the reducing agent, respectively, or mixing and adding the albumin and the reducing agent, in a storage container at an amount of one dose, and by adding samples (samples such as pond, fountain, water, etc) for detecting cadmium to the storage container at a predetermined amount.

Further, in accordance with still another aspect of the present invention, there is provided a kit for detecting cadmium including: the composition for detecting cadmium as described above.

Specifically, the kit for detecting the cadmium includes the detection composition, wherein a chamber is not specifically limited as long as it has a reaction space at which the sample and the detection composition are mixed, but may further include a heating means, a fluorescent light source, a fluorescence detector, a display part, etc., if needed.

An exemplary embodiment of the kit for detecting the cadmium including all of the above-described components is specifically illustrated in A of FIG. 15, and referring to this, the kit for detecting the cadmium may be constituted as follows.

The kit for detecting the cadmium 100 includes:

a chamber 110 having a reaction space;

a heating means 120 positioned below the chamber 110 and heating the chamber 110;

a light source 130 spaced apart from a left side of the chamber 110; and

a fluorescence detector 140 spaced apart from a right side of the chamber 110.

The kit for detecting the cadmium may further include a display part 150 electrically connected to the fluorescence detector 140 and positioned outside of the kit for detecting the cadmium 100, which is specifically illustrated in B of FIG. 15.

The kit for detecting the cadmium may further include a power supply, wherein the power supply is possible to be provided with external outlet or be charged, such that power is supplied by voltage drop or rectification so as to be appropriate for respective devices of the kit for detecting the cadmium. The power supply of the present invention is capable of using chargeable Li-ion battery cells that are charged by using an alternating current of 90V to 120V while supplying DC 12V, such that portability is easy.

In the display part 150, fluorescence detection values derived from the fluorescence detector 140 may be exhibited, or concentration of cadmium ions calculated from the values may be exhibited.

In addition, the display part 150 is supplied in an LCD device using an LCD module and separate charge-drop devices, and is designed to achieve control and monitoring directly through a display in a touch mode without input devices such as a mouse, a keyboard, etc.

Another kit for detecting cadmium according to the present invention is a device including the chamber 110 having the reaction space at which the composition for detecting cadmium is mixed with the sample to analyze cadmium concentration through a series of processes in which light in a ultraviolet ray region is irradiated from the light source 130, the light passed through a solution (reaction space) in which the composition for detecting cadmium is mixed with the sample is transferred to the fluorescence detector 140 again, and the fluorescence intensity of the cadmium cluster formed by mixing the composition for detecting cadmium with the sample is measured and analyzed.

Therefore, in the chamber 110, input and output parts in which light is irradiated are manufactured of quartz to have excellent transmission of the light source.

The chamber 110 may have a light transmission distance of about 0.5 to 4 mm.

The light source 130 may be a device for irradiating the fluorescent light source at an ultraviolet wavelength range in order to obtain the fluorescence spectrum from the cadmium nanocluster formed by mixing the sample with the detection composition in the reaction space of the chamber 110, wherein the light source 130 is not limited as long as it is a device capable of providing the light source of 350 to 370 nm, but may be preferably an UV lamp to measure cadmium concentration without difficulty in the field.

The fluorescence detector 140 is a detecting means capable of detecting fluorescence generated by irradiation of ultraviolet ray from the light source 130 to the chamber 110.

The fluorescence detector 140 is constituted to be capable of being measured with an interval of 1 nm to 2 nm.

The heating means 120 is not specifically limited as long as it is a device having a small volume so that the device is easily transported, and performing a heating process at 45 to 60° C., but preferably, may be a plurality of heating means including a blue LED (light emitting diode) element or a PN semiconductor that emits a heating light toward the reaction space of the chamber 110, which is the most preferred since it is capable of heating the sample regardless of kinds thereof in a uniform temperature distribution, and transportation and storage are easy due to a small volume.

The heating means may be provided with a control device for controlling or an alarm device when a temperature is more than 60° C.

In addition, another exemplary embodiment of the kit for detecting the cadmium is specifically illustrated in A of FIG. 16, and referring to this, the kit for detecting the cadmium may be constituted as follows.

The kit for detecting the cadmium 200 includes:

a chamber 210 having a reaction space;

a light source 220 positioned at a side of the chamber 210; and

a fluorescence detector 230 positioned to face the light source 220, having the chamber 210 interposed therebetween.

The kit for detecting the cadmium may further include a display part 240 electrically connected to the fluorescence detector 230 and positioned outside of the kit for detecting the cadmium 200, which is specifically illustrated in B of FIG. 16.

In the display part 240, fluorescence detection values derived from the fluorescence detector 230 may be exhibited, or concentration of cadmium ions calculated from the values may be exhibited.

In addition, the display part 240 is supplied in an LCD device using an LCD module and separate charge-drop devices, and is designed to achieve control and monitoring directly through a display in a touch mode without input devices such as a mouse, a keyboard, etc.

The kit for detecting the cadmium may further include a power supply, wherein the power supply is possible to be provided with external outlet or be charged, such that power is supplied by voltage drop or rectification so as to be appropriate for respective equipments of the kit for detecting the cadmium. The power supply of the present invention is capable of using chargeable Li-ion battery cells that are charged by using an alternating current of 90V to 120V while supplying DC 12V, such that portability is easy. Another kit for detecting cadmium according to the present invention is a device including the chamber 210 having the reaction space at which the composition for detecting cadmium is mixed with the sample to analyze cadmium concentration through a series of processes in which light in a ultraviolet ray region is irradiated from the light source 220, the light passed through a solution (reaction space) in which the composition for detecting cadmium is mixed with the sample is transferred to the fluorescence detector 230 again, and the fluorescence intensity of the cadmium cluster formed by mixing the composition for detecting cadmium with the sample is measured and analyzed.

Therefore, in the chamber 210, input and output parts in which light is irradiated are manufactured of quartz to have excellent transmission of the light source.

In addition, the chamber 210 may have a light transmission distance of about 0.5 to 4 mm.

The light source 220 may irradiate a fluorescent light in a ultraviolet wavelength range, and in addition thereto, may emit a heating light toward the reaction space of the chamber 110 in order to obtain the fluorescence spectrum from the cadmium nanocluster formed by mixing the sample with the detection composition in the reaction space of the chamber 210.

The light source 130 may consist of only a first light source that is capable of providing a light source at 350 to 370 nm, or may consist of the first light source that is capable of providing a light source at 350 to 370 nm and a second light source that emits the heating light.

The first light source is not specifically limited as long as it is a light source capable of providing the light source at 350 to 370 nm, but may preferably consist of a UV lamp to measure the cadmium concentration without difficulty in the field.

The second light source is not specifically limited as long as it is a light source capable of emitting the heating light for performing a heating process at 45 to 60° C., but preferably, may be a blue LED (light emitting diode) element, which may heat the sample regardless of kinds thereof in a uniform temperature.

The heating means may be provided with a control device for controlling or an alarm device (not illustrated) when a temperature is more than 60° C.

The fluorescence detector 230 is a detecting means capable of detecting fluorescence generated from irradiation of ultraviolet ray from the light source 220 to the chamber 210.

The albumin may be solid-state powder.

The reducing agent may be solid-state NaOH.

Hereinafter, the present invention will be described in detail through the following Examples; however, it is not construed as limiting the scope or the spirit of the present invention. In addition, as long as a person skilled in the art practices the present invention based on the disclosed description of the present invention including the following examples, it is obvious that the present invention may be easily practiced by a person skilled in the art even though testing results are not specifically provided.

Further, experimental results provided below mean representative experimental results of Examples and Comparative Examples, and respective effects of various exemplary embodiments of the present invention that are not explicitly described below are specifically described in corresponding parts.

<Material and Instrument>

<Material>

Bovine serum albumin (50 mg/ml) purchased from Fitzgerald (U.S.A.) was used. Human serum albumin (50 mg/ml) and ovalbumin (50 mg/ml) purchased from Wako Pure Chemical Industries, Ltd., were used.

The reducing agent was used by preparing 1 M NaOH solution, wherein NaOH was purchased from Sigma-Aldrich.

A sample containing cadmium was 10 mM cadmium chloride dihydrate (CdCl₂.2H₂O) purchased from Sigma-Aldrich.

<Instrument>

An incubated shaker was SI-300R (Jeiotech, Korea). In addition, a fluorescence spectrum measurement device was TECAN, and fluorescence images were obtained by using a ChemiDoc system (Bio-rad), and fluorescence intensities were analyzed by using a ChemiDoc program (Image Lab Software cersion 4.0, U.S.A.) from the obtained fluorescence images.

### EXAMPLE

For comparison in fluorescence intensities derived depending on whether albumin, the reducing agent, or cadmium is present in performing the method for detecting cadmium according to the present invention, Examples were prepared and tested as shown in Table 1 below.

TABLE 1

| | | | Sample |
|---|---|---|---|
| | | | 500 μl |
| | Detection composition | | (Concentration |
| | Albumin | Reducing agent | (mM) of |
| | 500 μl | 50 μl | Cadmium |
| NO. | (kind) | (kind) | chloride) |
| Example 1 | Distilled water | Distilled water | 10 mM |
| Example 2 | Distilled water | 1M NaOH | 10 mM |
| Example 3 | Human serum albumin | Distilled water | Distilled water |
| | 50 mg/ml | | |
| Example 4 | Human serum albumin | 1M NaOH | Distilled water |
| | 50 mg/ml | | |
| Example 5 | Human serum albumin | Distilled water | 10 mM |
| | 50 mg/ml | | |
| Example 6 | Human serum albumin | 1M NaOH | 10 mM |
| | 50 mg/ml | | |
| Example 7 | Distilled water | Distilled water | 10 mM |
| Example 8 | Distilled water | 1M NaOH | 10 mM |
| Example 9 | Ovalbumin | Distilled water | Distilled water |
| | 50 mg/ml | | |
| Example 10 | Ovalbumin | 1M NaOH | Distilled water |
| | 50 mg/ml | | |
| Example 11 | Ovalbumin | Distilled water | 10 mM |
| | 50 mg/ml | | |
| Example 12 | Ovalbumin | 1M NaOH | 10 mM |
| | 50 mg/ml | | |
| Comparative | Distilled water | Distilled water | 10 mM |
| Example 1 | | | |
| Comparative | Distilled water | 1M NaOH | 10 mM |
| Example 2 | | | |
| Comparative | Human serum albumin | Distilled water | Distilled water |
| Example 3 | 50 mg/ml | | |
| Comparative | Human serum albumin | 1M NaOH | Distilled water |
| Example 4 | 50 mg/ml | | |
| Comparative | Human serum albumin | Distilled water | 10 mM |
| Example 5 | 50 mg/ml | | |
| Comparative | Human serum albumin | 1M NaOH | 10 mM |
| Example 6 | 50 mg/ml | | |

### Test Example

### Test Example 1

### Detection of Cadmium from Samples Using Human Serum Albumin and Reducing Agent

Detection was performed by the following method using the detection compositions and the samples according to Examples 1 to 6, respectively, and respective fluorescence spectra resulted therefrom were obtained. The samples were mixed with the respective detection compositions at 37° C. and 300 rpm, and after 2 minutes, the reducing agent was added thereto and mixed with each other. Then, the respective mixtures were reacted under conditions of 37° C. and 300 rpm for 12 hours by using an incubated shaker, SI-300R (Jeiotech, Korea), and irradiated with UV, and fluorescence spectra thereof were measured.

As described above, fluorescence images obtained by mixing the detection compositions with the samples according to Examples 1 to 6 were analyzed, and fluorescence intensities derived therefrom were illustrated in FIG. 2.

As illustrated in FIG. 2, the fluorescence intensities were compared depending on whether cadmium ions, that is, cadmium chloride, human serum albumin, or NaOH is present, such that high fluorescence intensity of about 8000 a.u could be confirmed only in Example 6 using the detection composition (albumin and reducing agent) according to the present invention. As the cadmium nanocluster was formed, the fluorescence intensity of Example 6 was higher than those of other Examples, and it could be appreciated that whether the cadmium is present and concentration of cadmium could be confirmed only through the detection process according to the present invention.

In addition, it could be confirmed from Example 4 that when cadmium was not present in the sample, fluorescence was not exhibited even though the detection composition was mixed. That is, it could be appreciated that the detection method using the detection composition according to the present invention was selectively reacted with cadmium, and through this reaction, the cadmium nanocluster was formed. In Examples 3 to 5, it could be confirmed that fluorescence intensity was slightly increased due to fluorescence generated from residue of tryptophan present in albumin of the detection composition; however, this fluorescence intensity was lower by 2.5 times or more than fluorescence intensity increased in the presence of cadmium, which does not affect in detecting cadmium at all.

### Test Example 2

### Detection of Cadmium from Samples Using Ovalbumin and Reducing Agent

Detection was performed by the following method using the detection compositions and the samples according to Examples 7 to 12, respectively, and respective fluorescence spectra resulted therefrom were obtained. The samples were mixed with the respective detection compositions at 37° C. and 300 rpm, and after 2 minutes, the reducing agent was added thereto and mixed with each other. Then, the respective mixtures were reacted under conditions of 37° C. and 300 rpm for 12 hours by using an incubated shaker, SI-300R (Jeiotech, Korea), and irradiated with UV, and fluorescence images thereof were analyzed.

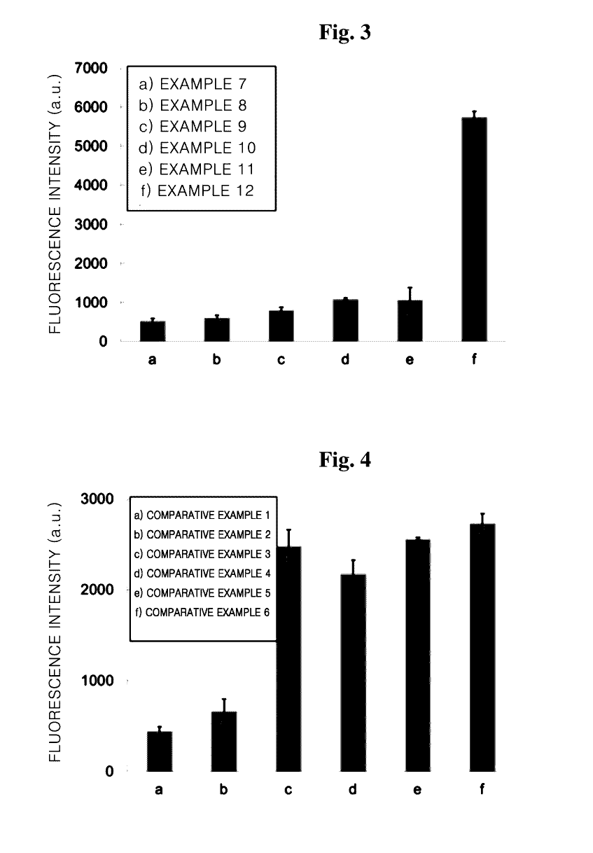

As described above, fluorescence images obtained by mixing the detection compositions with the samples according to Examples 7 to 12 were analyzed, and fluorescence intensities derived therefrom were illustrated in FIG. 3.

As illustrated in FIG. 3, the fluorescence intensities were compared depending on whether cadmium ions, that is, cadmium chloride, ovalbumin (OVA), or NaOH is present, such that it was confirmed that even though the fluorescence intensities obtained by using ovalbumin were lower than those obtained by using human serum albumin (HSA) in FIG. 2, similar patterns were exhibited.

Specifically, high fluorescence intensity of about 6000 a.u could be confirmed only in Example 12 using the detection composition including all components according to the present invention, which could be appreciated that as the cadmium nanocluster was formed by the detection composition according to the present invention, the fluorescence intensity of Example 12 was higher that those of Examples 7 to 11. From these results, it could be appreciated that whether the cadmium is present in the sample and concentration of cadmium could be derived through the detection method using the detection composition according to the present invention.

In addition, it could be appreciated from Example 10 in which cadmium is not present that high fluorescence was not exhibited only by the detection composition of the present invention, such that the detection method according to the present invention was selectively reacted with cadmium, and through the reaction of albumin and cadmium, the cadmium nanocluster was formed, thereby having high sensitivity. Meanwhile, in Examples 9 to 11, fluorescence intensity was slightly increased due to fluorescence generated from residue of tryptophan present in albumin; however, this fluorescence intensity was lower by 5 times or more than fluorescence intensity increased in the presence of cadmium, which does not affect in detecting cadmium at all.

It could be confirmed that the fluorescence intensity of the cadmium nanocluster formed by using the ovalbumin was lower by 1.5 to 2 times than that of the cadmium nanocluster formed by using the human serum albumin, and it was confirmed that sensitivity of the cadmium nanocluster formed by using the ovalbumin was much lower than that of the cadmium nanocluster formed by using the human serum albumin.

However, in the case of using ovalbumin, sensitivity and detection time are long, and the fluorescence intensity is slightly low, but it is usable for qualitative detection of cadmium due to a low cost, such that ovalbumin or human serum albumin may be appropriately selected depending on uses and places.

### Test Example 3

### Detection of Cadmium from Samples Using Bovine Serum Albumin and Reducing Agent

Detection was performed by the following method using the detection compositions and the samples according to Comparative Examples 1 to 6, respectively, and respective fluorescence images resulted therefrom were obtained. The samples were mixed with the respective detection compositions at 37° C. and 300 rpm, and after 2 minutes, the reducing agent was added thereto and mixed with each other. Then, the respective mixtures were reacted under conditions of 37° C. and 300 rpm for 12 hours by using an incubated shaker, SI-300R (Jeiotech, Korea), and irradiated with UV, and fluorescence images thereof were measured.

As described above, fluorescence images obtained by mixing the detection compositions with the sample according to Comparative Examples 1 to 6 were analyzed, and fluorescence intensities derived therefrom were illustrated in FIG. 4.

As illustrated in FIG. 4, the fluorescence intensities were compared depending on whether cadmium ions, that is, cadmium chloride, bovine serum albumin (BSA), or NaOH is present, and results thereof were different from those of FIGS. 2 and 3.

Specifically, all of Comparative Examples 3 to 6 exhibited fluorescence intensities of about 2000 to 3000 a.u.

That is, there was no significant difference between Comparative Example 6, and Comparative Examples 3 to 5 in view of fluorescence intensity, such that it could be appreciated that the bovine serum albumin was not capable of forming the cadmium nanocluster by the reaction with cadmium ions.

In particular, Comparative Examples 3 to 6 exhibited the fluorescence intensities similar to those of Examples 3 and 9 to which only albumin is added, from which could be appreciated that the fluorescence intensities thereof were measured from residue tryptophan present in bovine serum albumin. In other words, it could be appreciated that the bovine serum albumin was not reacted with cadmium ions.

### Test Example 4

### Detection of Cadmium Using Human Serum Albumin and Reducing Agent with Respect to Samples for Each Concentration of Cadmium Ions

Fluorescence intensities were derived by mixing the detection compositions with the samples shown in Table 2 below. For the detection process, specifically, the samples were mixed with albumin at 37° C. and 300 rpm, and after 2 minutes, the reducing agent was added thereto and mixed with each other. Then, the respective mixtures were reacted under conditions of 37° C. and 300 rpm for 12 hours by using an incubated shaker, SI-300R (Jeiotech, Korea), and irradiated with UV, and fluorescence images thereof were measured.

TABLE 2

| | | | Sample |
|---|---|---|---|
| | | | 500 μl |
| | Detection composition | | (Concentration |
| | Albumin | Reducing agent | (nM) of |
| | 500 μl | 50 μl | Cadmium |
| NO. | (kind) | (kind) | chloride) |
| Example 13 | Human serum | 1M NaOH | 10⁻³ |
| | albumin | | |
| | 50 mg/ml | | |
| Example 14 | Human serum | 1M NaOH | 10⁻² |
| | albumin | | |
| | 50 mg/ml | | |
| Example 15 | Human serum | 1M NaOH | 10⁻¹ |
| | albumin | | |
| | 50 mg/ml | | |
| Example 16 | Human serum | 1M NaOH | 10⁻⁰ |
| | albumin | | |
| | 50 mg/ml | | |
| Example 17 | Human serum | 1M NaOH | 10¹ |
| | albumin | | |
| | 50 mg/ml | | |
| Example 18 | Human serum | 1M NaOH | 10² |
| | albumin | | |
| | 50 mg/ml | | |
| Example 19 | Human serum | 1M NaOH | 10³ |
| | albumin | | |
| | 50 mg/ml | | |
| Example 20 | Human serum | 1M NaOH | 10⁴ |
| | albumin | | |
| | 50 mg/ml | | |

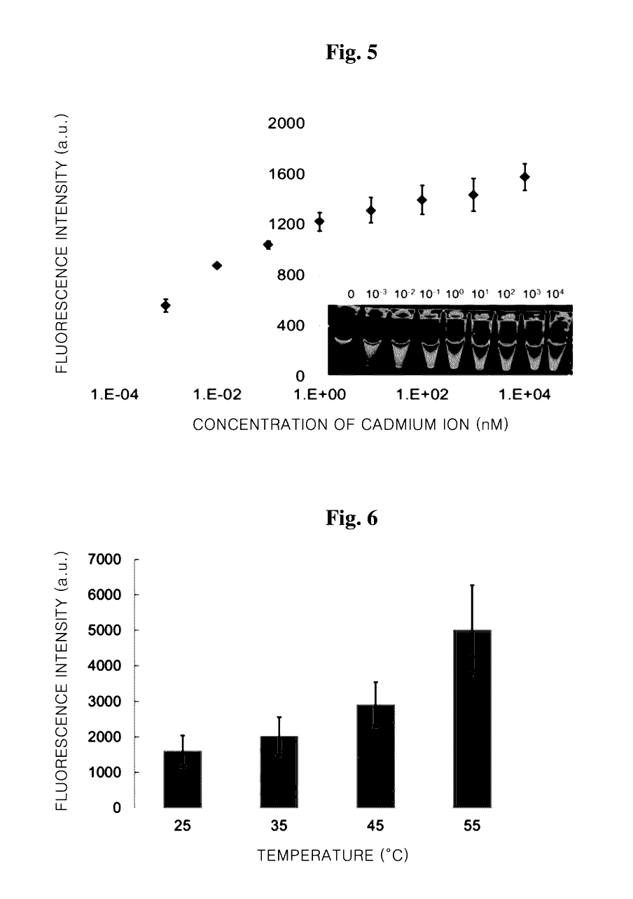

As described above, respective fluorescence images obtained by mixing the detection composition with the samples according to Examples 13 to 20 were analyzed, and fluorescence intensities derived therefrom were illustrated in FIG. 5.

As illustrated in FIG. 5, it was confirmed that as a concentration of the cadmium ions in the sample mixed with the detection composition of the present invention was increased, the derived fluorescence intensity was also increased. It could be appreciated from these results that as the concentration of the cadmium ions was increased, a diameter of the cadmium nanocluster formed by combining with albumin was increased, such that the fluorescence intensity dependent on the cadmium nanocluster was also increased.

Therefore, it could be appreciated that the detection composition according to the present invention and the detection method using the detection composition are used to confirm whether or not the cadmium ions are present in the sample and the concentration of cadmium ions.

It indicated that it is possible to detect cadmium present in the sample quantitatively and qualitatively by using the detection method and the detection kit according to the present invention.

### Test Example 5

### Detection Temperature Condition for Decreasing Detection Time

Various conditions (temperature, reaction time, and concentration of reducing agent) were confirmed in the detection process in order to decrease the reaction time, specifically, 12 hours applied for Test Examples 1 to 4. When cadmium is detected under these conditions, time for detection may be remarkably decreased as compared to the existing protein directed synthesis method.

Detection was performed by the following method using the detection composition and the sample according to Example 6, respectively, and respective fluorescence images resulted therefrom were obtained.

The sample was mixed with albumin at 37° C. and 300 rpm, and after 2 minutes, the reducing agent was added thereto and mixed with each other. Then, the mixture was reacted at 300 rpm for 3 hours under various temperature conditions by using an incubated shaker, SI-300R (Jeiotech, Korea), and irradiated with UV, and fluorescence images thereof were measured. Here, the respective experiments were performed at the temperature conditions of 25° C., 35° C., 45° C. and 55° C.

As described above, the respective fluorescence images obtained by mixing the detection composition with the sample according to Example 6 under various temperatures were analyzed, and fluorescence intensities derived therefrom were illustrated in FIG. 6.

As illustrated in FIG. 6, in order to decrease the detection time in the detection method according to the present invention, the step of obtaining the cadmium nanocluster formed by the reaction of albumin and cadmium ions could be optimized, such that the cadmium nanocluster is rapidly formed, thereby decreasing the detection time.

Therefore, to sum the results of FIG. 6 up in addition to the above description, it could be confirmed that when cadmium was detected at 55° C. among temperature conditions of 25° C. to 55° C., the fluorescence intensity was the highest. It indicated that when a temperature was 55° C. under the same detection conditions, the reaction of albumin and cadmium was rapidly performed, such that the cadmium nanocluster was rapidly manufactured. Specifically, it could be confirmed that the fluorescence intensity at 55° C. was higher by about 2 times than the fluorescence intensity at 25° C.

### Test Example 6

### Concentration Condition of Reducing Agent for Decreasing Detection Time

Fluorescence intensities were derived by mixing the detection compositions with samples shown in Table 3 below. For the detection process, specifically, the samples were mixed with albumin at 55° C. and 300 rpm, and after 2 minutes, the reducing agent was added thereto and mixed with each other. Then, the mixture was reacted at 55° C. and 300 rpm for 2 hours by using an incubated shaker, SI-300R (Jeiotech, Korea), and irradiated with UV, and fluorescence images thereof were measured.

As described above, the respective fluorescence images obtained by mixing the detection compositions of Example 21 to 27 with the samples were analyzed, and fluorescence intensities derived therefrom were illustrated in FIG. 7.

TABLE 3

| | | | Sample |
|---|---|---|---|
| | | | 500 μl |
| | Detection composition | | (Concentration |
| | Albumin | Reducing agent | (mM) of |
| | 500 μl | 50 μl | Cadmium |
| NO. | (kind) | (kind) | chloride) |
| Example 21 | Human serum | 0M NaOH | 10 |
| | albumin | | |
| | 50 mg/ml | | |
| Example 22 | Human serum | 0.0625M NaOH | 10 |
| | albumin | | |
| | 50 mg/ml | | |
| Example 23 | Human serum | 0.125M NaOH | 10 |
| | albumin | | |
| | 50 mg/ml | | |
| Example 24 | Human serum | 0.25M NaOH | 10 |
| | albumin | | |
| | 50 mg/ml | | |
| Example 25 | Human serum | 0.5M NaOH | 10 |
| | albumin | | |
| | 50 mg/ml | | |
| Example 26 | Human serum | 1M NaOH | 10 |
| | albumin | | |
| | 50 mg/ml | | |
| Example 27 | Human serum | 2M NaOH | 10 |
| | albumin | | |
| | 50 mg/ml | | |

In order to reduce the detection time in the detection method according to the present invention, it is required to maximally secure the cadmium nanocluster exhibiting the fluorescence property in a short period of time by decreasing time for synthesizing the cadmium nanocluster through the reaction of albumin and cadmium ions.

As illustrated in FIG. 7, the cadmium sample was detected with the detection composition by changing concentrations of sodium hydroxide (NaOH) which is the reducing agent, and as a result, it could be appreciated that the fluorescence intensity of Example 25 using 0.5 M concentration of reducing agent (NaOH) was higher by about 2 times or more than that of Example 26 using 1 M concentration of reducing agent (NaOH).

It could be appreciated from the result that when the concentration of the reducing agent is 0.25 to 0.9 M in the detection composition according to the present invention, time required for detecting cadmium by using the detection composition of the present invention could be decreased by 4 times or more.

If the concentration of the reducing agent in the detection composition is lower than 0.25 M or higher than 0.9 M, since time for synthesizing the cadmium nanocluster through the reaction of cadmium and albumin is increased, the detection time is needed for 10 hours or more so as to induce sufficient synthesis, and if the detection time is shorter than 10 hours, sensitivity and accuracy are decreased.

### Test Example 7

### Confirmation of Optimum Detection Time Depending on Detection Conditions

Detection properties of cadmium depending on time were confirmed under various conditions by using the detection composition and the sample according to Example 25.

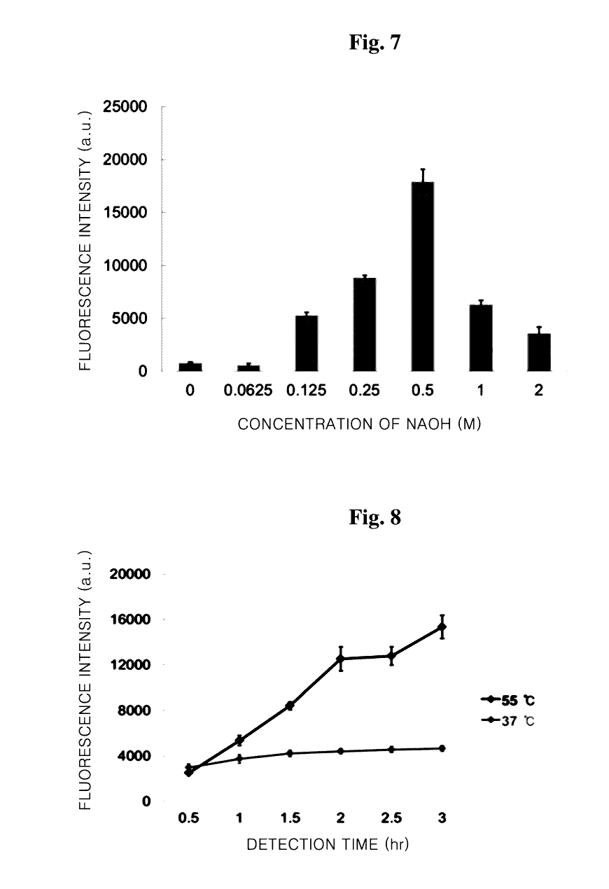

Specifically, the sample was mixed with the detection composition at 55° C. and 300 rpm, and after 2 minutes, the reducing agent was added thereto and mixed with each other. Then, the mixture was reacted under conditions of 55° C. and 300 rpm for 55 hours by using an incubated shaker, SI-300R (Jeiotech, Korea) while changing reaction time, and irradiated with UV, and fluorescence spectra thereof were measured. The reaction time was 0.5 hour, 1 hour, 1.5 hour, 2 hour, 2.5 hour and 3 hour, respectively. The results were marked with points of 55° C. in a graph of FIG. 8.

In addition, another test was performed in the same manner as the above test except that the temperature of the detection conditions is changed from 55° C. to 37° C., and results were compared with those of the above test. The results were marked with points of 37° C. in a graph of FIG. 8.

As illustrated in FIG. 8, when the sample was detected under determined conditions in Test Examples 5 and 6 (temperature and concentration of reducing agent), time for completely synthesizing the cadmium nanocluster formed by the reaction of albumin and cadmium ions could be confirmed.

First, when the detection was performed by using the detection composition having the optimized concentration of the reducing agent and the sample according to Example 25 at the optimum detection temperature of 55° C., the highest fluorescence intensity was exhibited from after 2 hours.

That is, according to the method for detecting cadmium of the present invention, albumin and cadmium in the sample were actively reacted under conditions of 55° C. and the concentration of reducing agent (0.5 M) to rapidly form the cadmium nanocluster, such that high fluorescence intensity was capable of being derived from after 2 hours.

On the contrary, when the detection was performed at 37° C., the highest fluorescence intensity was not observed. That is, even though the reaction time was increased, there was no significant difference in fluorescence intensity, and it was generally confirmed that the fluorescence intensity was about 4000 a.u or less, and accordingly, it could be appreciated that when the temperature is less than 40° C., a long period of time for 3 hours or more is required until the cadmium nanocluster is completely formed.

Consequently, in the case where the detection composition of the present invention is used to perform detection of cadmium, only when both of the temperature and the concentration of the reducing agent are satisfied, the reaction rate of albumin and cadmium is rapid, such that the fluorescence intensity may be derived within a short period of time. If any one of the above conditions is not satisfied, since the reaction rate of albumin and cadmium is significantly decreased, time for obtaining sufficient fluorescence intensity for deriving the cadmium concentration is more needed by 4 times or more.

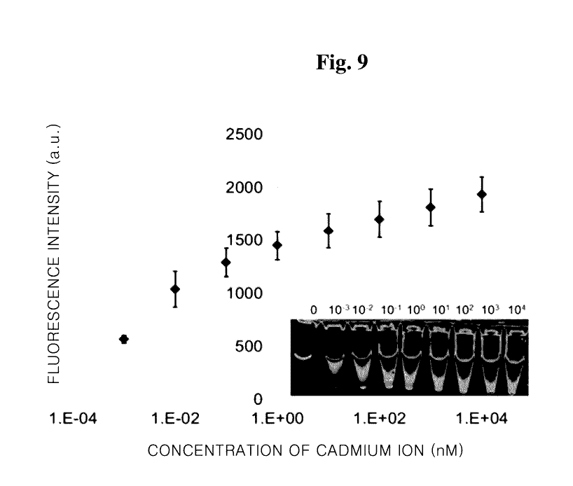

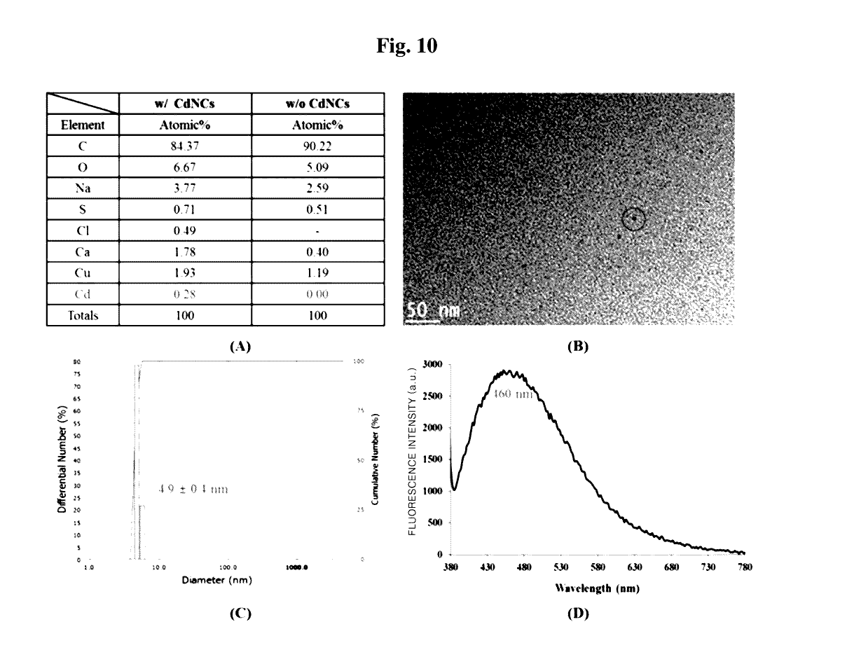

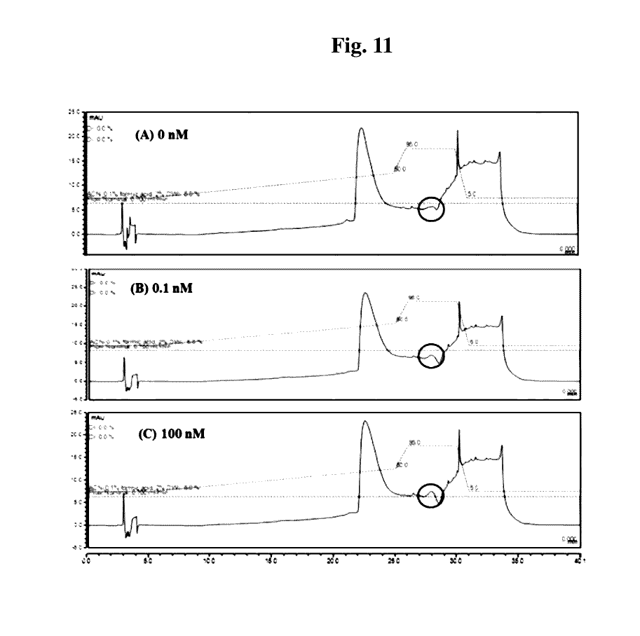

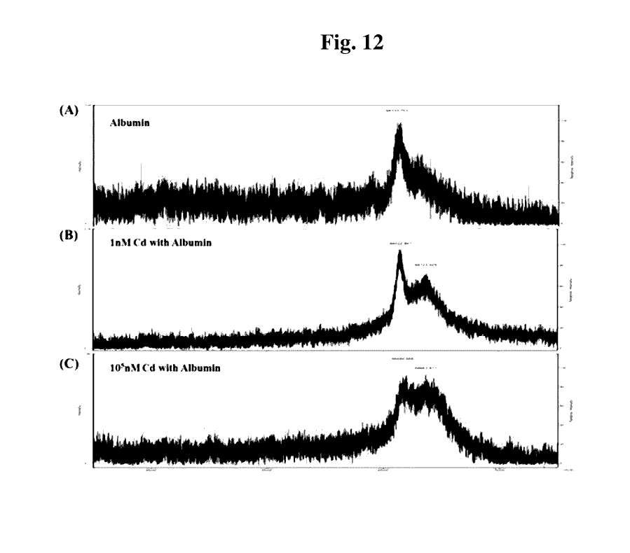

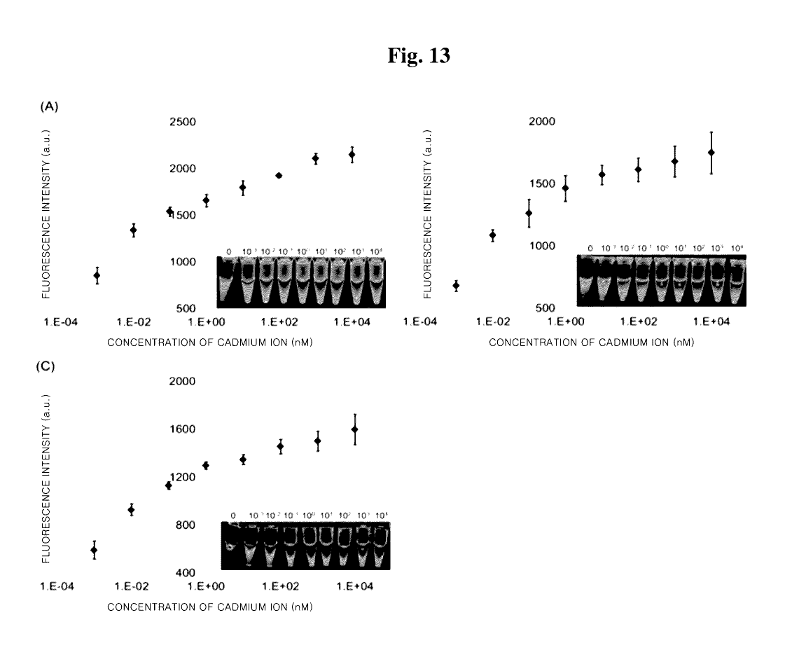

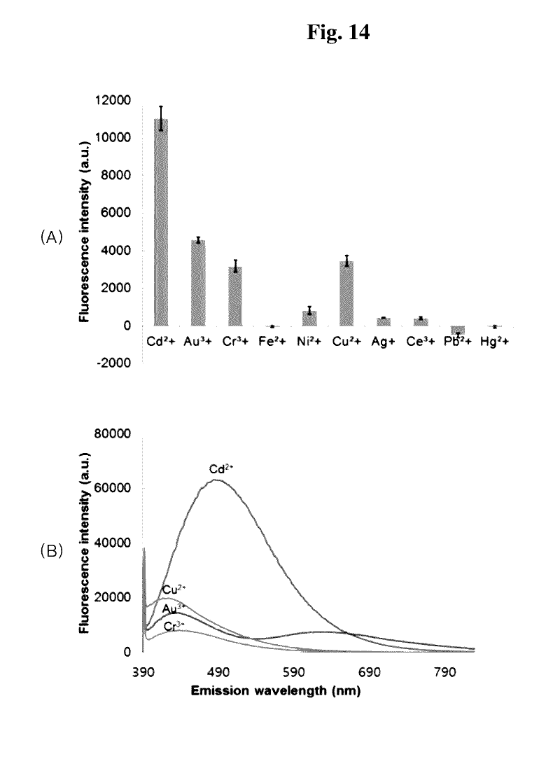

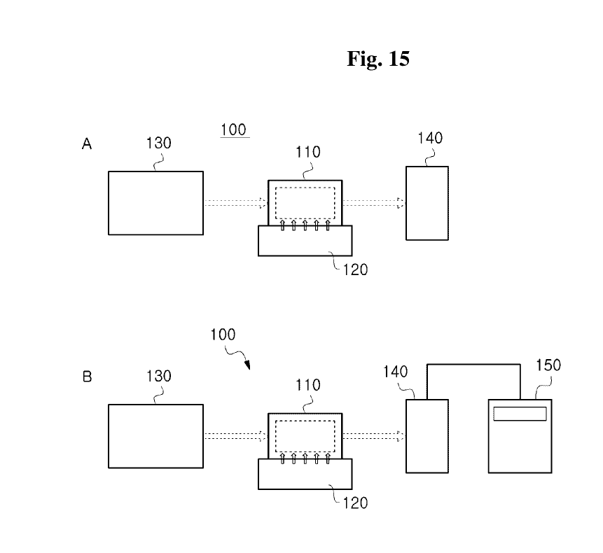

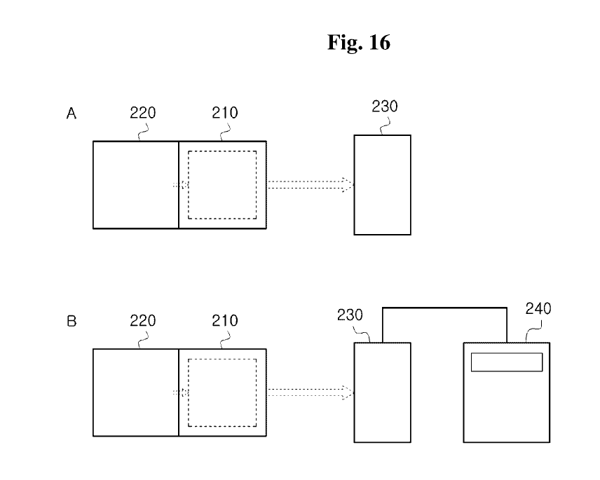

Samples including cadmium ions having various concentrations were prepared, and detection compositions each including human serum albumin 50 mg/ml and 0.5 M NaOH were mixed with each sample, and fluorescence spectra of the respective mixtures were obtained. Fluorescence intensities were analyzed from the obtained fluorescence spectra, and illustrated in FIG. 9. The samples and the detection compositions used therein were shown in Table 4 below in detail.

TABLE 4

| | | | Sample |
|---|---|---|---|
| | Detection composition | | 500 μl |
| | | Reducing | (Concentration |
| | Albumin | agent | (nM) of |
| | 500 μl | 50 μl | Cadmium |
| NO. | (kind) | (kind) | ions) |
| Example 28 | Human serum | 0.5M NaOH | 10⁻³ |
| | albumin | | |
| | 50 mg/ml | | |
| Example 29 | Human serum | 0.5M NaOH | 10⁻² |
| | albumin | | |
| | 50 mg/ml | | |
| Example 30 | Human serum | 0.5M NaOH | 10⁻¹ |
| | albumin | | |
| | 50 mg/ml | | |
| Example 31 | Human serum | 0.5M NaOH | 10⁻⁰ |
| | albumin | | |
| | 50 mg/ml | | |
| Example 32 | Human serum | 0.5M NaOH | 10¹ |
| | albumin | | |
| | 50 mg/ml | | |
| Example 33 | Human serum | 0.5M NaOH | 10² |
| | albumin | | |
| | 50 mg/ml | | |
| Example 34 | Human serum | 0.5M NaOH | 10³ |
| | albumin | | |
| | 50 mg/ml | | |
| Example 35 | Human serum | 0.5M NaOH | 10⁴ |
| | albumin | | |
| | 50 mg/ml | | |

Specifically, the respective samples were mixed with albumin at 55° C. and 300 rpm, and after 2 minutes, the reducing agent was added thereto and mixed with each other. Then, the mixtures were reacted under conditions of 55° C. and 300 rpm for 55 hours by using an incubated shaker, SI-300R (Jeiotech, Korea) while changing reaction time, and irradiated with UV, and fluorescence spectra thereof were measured and analyzed to derive fluorescence intensities. The derived fluorescence intensities were illustrated in FIG. 9. The reaction time was 2 hours.

As illustrated in FIG. 9, it could be appreciated that a slope of a graph depending on concentration of cadmium ions was steeper than that of a graph of FIG. 5. It indicated that when cadmium was detected under optimum detection condition, sensitivity and accuracy to cadmium were more excellent.

### Test Example 8

### Properties of Cadmium Cluster Formed by Detection Process

Detection properties of cadmium were confirmed through the following detection process using the detection composition and the sample including cadmium ions according to Example 25.

Specifically, the sample was mixed with the detection composition at 55° C. and 300 rpm, and after 2 minutes, the reducing agent was added thereto and mixed with each other. Then, the mixture was reacted under conditions of 55° C. and 300 rpm for 2 hours by using an incubated shaker, SI-300R (Jeiotech, Korea), and a detection solution of which the reaction is terminated was irradiated with UV, and light source passed through the detection solution of which the reaction is terminated was analyzed, thereby measuring fluorescence spectrum.

The detection solution of which the reaction is terminated means a solution in a state in which the cadmium cluster, the sample, and unreacted detection composition is mixed after the sample is mixed with the detection composition, and the reaction is completely terminated.

For comparison with this, a detection solution of which the reaction is terminated was prepared in the same manner as the above-described process except for using a sample in which cadmium is not present, which is regarded as a Control Group.

(A) in FIG. 10 is a Table illustrating analysis results of energy-dispersive X-ray spectroscopy (JEM-2100F manufactured by JEOL, Japan) between the detection solution of which the reaction is terminated and the Control Group obtained through the detection process of Test Example 8 according to the present invention using the sample in which cadmium ions are present and the sample in which cadmium ions are not present.

Here, w/CdNCs indicates the detection solution of which the reaction is terminated prepared by reacting the sample in which cadmium ions are present through the above-described process of Example 25, and w/o CdNC indicates the Control Group using the sample in which cadmium ions are not present.

As illustrated in (A) in FIG. 10, cadmium atoms were observed in the detection solution of which the reaction is terminated that detected the sample in which cadmium ions are present. On the contrary, the cadmium atoms were not observed in the Control Group using the sample in which cadmium ions are not present.

It could be appreciated from the results that the cadmium nanocluster was formed only through the combination of cadmium and albumin.

(B) in FIG. 10 is a Table illustrating an image of transmission electron microscopy (JEM-2100F manufactured by JEOL, Japan) of the detection solution of which the reaction is terminated obtained through the detection process of Test Example 8 according to the present invention using the sample in which cadmium ions are present.

As illustrated in (B) in FIG. 10, it could be confirmed that the cadmium cluster was present in the detection solution of which the reaction is terminated, and also confirmed that the cadmium cluster had a spherical shape and a diameter of about 5 nm. Meanwhile, the diameter of the cadmium nanocluster may vary depending on the concentration of cadmium ions in the sample in which cadmium ions are present.

(C) in FIG. 10 is a graph illustrating measurement results of dynamic light scattering (ELS-Z manufactured by Otsuka electronic, Japan) on the detection solution of which the reaction is terminated obtained through the detection process of Test Example 8 according to the present invention using the sample in which cadmium ions are present.

As illustrated in (C) in FIG. 10, it could be confirmed that the diameter of the cadmium nanocluster present in the detection solution of which the reaction is terminated was about 4.5 to 5.3 nm. The result is the same as that of the transmission electron microscopy of (B) in FIG. 10.

(D) in FIG. 10 is a graph illustrating measurement results of fluorescence emission spectra (TECAN manufactured by Mannedorf, Switzerland) on the detection solution of which the reaction is terminated obtained through the detection process of Test Example 8 according to the present invention using the sample in which cadmium ions are present.

As illustrated in (D) in FIG. 10, it was confirmed that the detection solution of which the reaction is terminated had an absorption wavelength of 365 nm and an emission wavelength of 460 nm by the cadmium nanocluster formed by the reaction. It could be appreciated from the above result that the cadmium nanocluster exhibiting fluorescence under UV was formed.

In addition, it could be appreciated that fluorescence images could be obtained from the detection solution of which the reaction is terminated by using a fluorescence image measurement equipment (Chemi-doc, manufacturer: Bio-rad (U.S.A.), Model name: Universal Hood III) and analysis program (Image lab software version 4.0) under UV, and fluorescence intensities thereof could be measured and analyzed.

FIG. 11 is a graph illustrating high performance liquid chromatography (HPLC) analysis results on the detection solution of which the reaction is terminated obtained in the same manner as the detection process of Test Example 8 except for using samples each having different concentration of cadmium ions in order to confirm whether the cadmium nanocluster formed in the detection solution of which the reaction is terminated obtained by the detection process of Test Example 8 is affected depending on the concentration of cadmium ions.

This experiment was performed in the same manner except that the concentrations of cadmium ions present in the sample were 0 nM, 0.1 nM and 100 nM in the detection process. The detection solutions of which the reaction is terminated each having different concentration of cadmium nanocluster were obtained by mixing only the detection composition (Test Example 25) with the samples each having different concentration of cadmium ions, and analyzed by HPLC.

Here, A in FIG. 11 is a case in which the concentration of cadmium ions in the sample in which cadmium ions are present is 0 nM, B in FIG. 11 is a case in which the concentration of cadmium ions is 0.1 nM, and C in FIG. 11 is a case in which the concentration of cadmium ions is 100 nM.

As illustrated in FIG. 11, it could be confirmed that as the concentration of cadmium ions was increased, signal (illustrated in black circles) at a predetermined region in the HPLC graph was also increased, which indicated an amount of cadmium atoms present in the formed cadmium nanocluster, such that it could be appreciated that as the concentration of cadmium ions was increased, the diameter of the cadmium nanocluster was also increased.

FIG. 12 is a graph illustrating measurement results of MALDI-TOF MS (ASTA, KOREA) on the detection solution of which the reaction is terminated obtained in the same manner as the detection process of Test Example 8 except for using samples each having different concentration of cadmium ions in order to confirm whether the cadmium nanocluster formed in the detection solution of which the reaction is terminated obtained by the detection process of Test Example 8 is affected depending on the concentration of cadmium ions.

This experiment was performed in the same manner except that the concentrations of cadmium ions present in the sample were 0 nM, 0.1 nM and 100 nM in the detection process. The detection solutions of which the reaction is terminated each having different concentration of cadmium nanocluster were obtained by mixing only the detection composition (Test Example 25) with the samples each having different concentration of cadmium ions, and analyzed.

Here, (A) in FIG. 12 is a case in which the concentration of cadmium ions in the sample in which cadmium ions are present is 0 nM, (B) in FIG. 12 is a case in which the concentration of cadmium ions is 0.1 nM, and (C) in FIG. 12 is a case in which the concentration of cadmium ions is 100 nM.

As illustrated in FIG. 12, it was observed from the analysis result of MALDI-TOF MS on the formed cadmium nanocluster that as the cadmium ions were large in the sample, second signal of the graph was increased.

Upon comparing with the graph of FIG. 12 with a case in which cadmium is not present, it was confirmed that about 20 cadmium atoms were capsulated in the human serum albumin to form the cadmium nanocluster, which is calculated by dividing 2 kDa which is a degree of a shifted peak when cadmium is present, into a molecular weight of cadmium.

### Test Example 9

### Confirmation Whether Cadmium is Detected from Various Samples

FIG. 13 illustrates fluorescence images obtained by performing the method for detecting cadmium of the present invention on real samples such as tap water (A), fountain water (B), and pond water (C) under optimized conditions, and analysis results of fluorescence intensities thereof, wherein the fluorescence images and the fluorescence intensities of all graphs except for (D) in FIG. 10 were obtained by using a ChemiDoc system (Bio-rad).

Specifically, in the method for detecting cadmium, the real samples (500 μl) were mixed at 55° C. and 300 rpm with human serum albumin (500 μl) having a concentration of 50 mg/ml, and after 2 minutes, various concentrations of NaOH (50 μl) was added thereto and mixed with each other. Then, the mixtures were reacted at 55° C. and 300 rpm for various time by using an incubated shaker, SI-300R (Jeiotech, Korea), and irradiated with UV, and fluorescence spectra thereof were measured.

As illustrated in FIG. 13, it was confirmed that the tap water, the fountain water, and the pond water of the real samples had a limit of detection (LOD) of cadmium of 0.75 fM, 7.65 fM, and 48.2 fM, respectively, (see Table 5 below). It could be appreciated from the result that even in the real samples, the concentration of cadmium could be measured with significantly high sensitivity.

### Test Example 10

### Specificity of Method for Detecting Cadmium According to the Present Invention

In order to confirm that the detection method of the present invention has specificity to cadmium ions, the detection was performed under the following conditions.

Specifically, various samples (500 μl) including any one metal ion were mixed at 55° C. and 300 rpm with human serum albumin (500 μl) having a concentration of 50 mg/ml, and after 2 minutes, various concentrations of NaOH (50 μl) was added thereto and mixed with each other. Then, the mixtures were reacted at 55° C. and 300 rpm for various time by using an incubated shaker, SI-300R (Jeiotech, Korea), and irradiated with UV to obtain fluorescence spectra, and fluorescence intensities thereof were analyzed and illustrated in (A) and (B) in FIG. 14.

The metal ions were cadmium ion (Cd²⁺), gold ion (Au³⁺), chromium ion (Cr³⁺), iron ion (Fe²⁺), nickel ion (Ni²⁺), copper ion (Cu²⁺), silver ion (Ag⁺), cerium ion (Ce³⁺), palladium ion (Pb²⁺), mercury ion (Hg²⁺).

FIG. 14 is a graph illustrating analysis results of fluorescence intensities (a.u.) obtained by performing the method for detecting cadmium of the present invention on various samples including the metal ions through the above-described process in order to confirm specificity of the method for detecting cadmium of the present invention to the cadmium ions. (A) in FIG. 14 is a graph illustrating analysis results of fluorescence images obtained by using ChemiDoc system (Bio-rad), and (B) in FIG. 14 is a graph illustrating fluorescence spectra obtained by using TECAN on gold ion (Au³⁺), chromium ion (Cr³⁺), and copper ion (Cu²⁺) having fluorescence intensity weaker than that of the cadmium ion (Cd²⁺) but exhibiting some degree of fluorescence.

As illustrated in (A) and (B) in FIG. 14, in order to measure the specificity of the detection method of the present invention on various metal ions, various samples including the respective metal ions were detected, analyzed, and compared by the above-described method, and as a result, the fluorescence intensity from the sample including cadmium ions was stronger by about 4 to 10 times than those of the samples including different metal ions. It could be appreciated from the result that the detection method of the present invention had specificity, that is, selectivity, on the cadmium ions.

Therefore, it was confirmed that even in the samples in which various metal ions are present, the cadmium ions were selectively reacted with albumin used in the present detection method to form the cadmium nanocluster, such that it could be appreciated that when the detection method of the present invention is performed on real samples, it is possible to measure whether the cadmium ions are present and the concentration of cadmium ions with high sensitivity and selectivity.

### Test Example 11

### Accuracy on Method for Detecting Cadmium According to the Present Invention

In order to confirm that the detection method of the present invention has accuracy in detecting concentration of cadmium ions, the detection was performed under the following conditions.

Specifically, various samples (500 μl) including cadmium ions were mixed at 55° C. and 300 rpm with human serum albumin (500 μl) having a concentration of 50 mg/ml, and after 2 minutes, 0.5 M NaOH (50 μl) was added thereto and mixed with each other. Then, the mixtures were reacted at 55° C. and 300 rpm for 2 hours by using an incubated shaker, SI-300R (Jeiotech, Korea), and irradiated with UV to obtain fluorescence spectra, and limit of detection (LOD) calculated therefrom was shown in Table 5 below. The samples were prepared by mixing cadmium ions each having concentration of 10⁻³ nM, 10⁻² nM, 10⁻¹ nM, 10⁰ nM with tap water, fountain water, and pond water, respectively, as described above.

LOD may be calculated by Equation: LOD=3×S_{R}/m, wherein m is a slope of a calibration curve and S_{R} is a standard deviation.

Table 5 shows LOD calculation values on cadmium ions from various samples including the cadmium ions through the detection method of the present invention.

TABLE 5

| | Concentration | Detection composition | | |
|---|---|---|---|---|
| | (nm) of | | Reducing | |
| Kinds of | Cadmium (cd²⁺) | Albumin | agent | Limit of |
| sample | added | 500 μl | 50 μl | detection |
| 500 μl | in sample | (kind) | (kind) | (lod) |
| Tap water | 10⁻³ | Human | 0.5 m naoh | 0.75 fm |
| | 10⁻² | serum | | |
| | 10⁻¹ | albumin | | |
| | 10⁰ | 50 mg/ml | | |
| Fountain | 10⁻³ | | | 7.65 fm |
| water | 10⁻² | | | |
| | 10⁻¹ | | | |
| | 10⁰ | | | |
| Pond water | 10⁻³ | | | 48.2 fm |
| | 10⁻² | | | |
| | 10⁻¹ | | | |
| | 10⁰ | | | |

As shown in Table 5, it was confirmed that the method for detecting cadmium of the present invention had LOD of 0.75 fM, 7.65 fM, and 48.2 fM.

The method for detecting cadmium of the present invention has a lower LOD than an internationally accepted tolerance range of cadmium, such that it is possible to detect the cadmium concentration even in a non-toxic range from various samples (tap water, fountain water, pond water, etc), thereby directly detecting the concentration of cadmium ions with high sensitivity.

Specifically, various samples (500 μl) including cadmium ions were mixed at 55° C. and 300 rpm with human serum albumin (500 μl) having a concentration of 50 mg/ml, and after 2 minutes, 0.5 M NaOH (50 μl) was added thereto and mixed with each other. Then, the mixtures were reacted at 55° C. and 300 rpm for 2 hours by using an incubated shaker, SI-300R (Jeiotech, Korea), and irradiated with UV to obtain fluorescence spectra. From the obtained fluorescence spectra, cadmium concentration was measured and accuracy was calculated, and results were shown in Table 6 below. The samples were prepared by mixing cadmium ions each having concentration of 0.5 nM, 1 nM, and 5′ nM with tap water, fountain water, and pond water, respectively, as described above.

Here, accuracy (%) is calculated as follows: a measured concentration of cadmium/a concentration of cadmium added in sample×100(%)

TABLE 6

| | Concen- | Detection | | Measured | |
|---|---|---|---|---|---|
| | tration | composition | | concen- | |
| Kinds | (nm) of | | Reducing | tration | |
| of | Cadmium | Albumin | agent | (nm) of | |
| sample | (cd²⁺) added | 500 μl | 50 μl | cadmium | Accuracy |
| 500 μl | in sample | (kind) | (kind) | (cd²⁺) | (%) |
| Tap | 0.5 | Human | 0.5 m | 0.486 | 97.15 |
| water | 1 | serum | naoh | 1.006 | 100.56 |
| | 5 | albumin | | 5.494 | 109.88 |
| Fountain | 0.5 | 50 mg/ml | | 0.437 | 87.47 |
| water | 1 | | | 0.810 | 81.04 |
| | 5 | | | 4.915 | 98.30 |
| Pond | 0.5 | | | 0.528 | 105.67 |
| water | 1 | | | 0.975 | 97.45 |
| | 5 | | | 4.681 | 93.62 |

As shown in Table 6, it could be appreciated that as a result obtained by detecting ions through the detection method of the present invention as compared to cadmium concentration known in the art, high accuracy (%) of 80 to 110% was achieved. Specifically, it could be confirmed that the concentration of cadmium ions could be detected with high accuracy without being affected by various materials present in real samples such as tap water, fountain water, and pond water.

According to various exemplary embodiments of the present invention, the method for detecting cadmium according to the present invention may easily, rapidly, and simply detect the cadmium quantitatively and qualitatively by sequentially adding the albumin and the reducing agent to the sample, the albumin and the reducing agent inducing synthesis of the cadmium nanocluster capable of measuring a change in fluorescence intensity.

In addition, the albumin and the reducing agent used for the method for detecting cadmium have excellent sensitivity even though they are low-cost, such that the concentration of the cadmium present in the sample is capable of being directly measured.

Although some embodiments have been disclosed herein, it should be understood by those skilled in the art that these embodiments are provided by way of illustration only, and that various modifications, changes, and alterations can be made without departing from the spirit and scope of the invention. Therefore, it should be understood that the foregoing embodiments are provided for illustrative purposes only and are not to be construed in any way as limiting the present invention.

1. A method for detecting cadmium, comprising:
I) mixing and reacting a detection composition including albumin and a reducing agent with a sample containing cadmium to obtain a cadmium nanocluster; and
II) irradiating the cadmium nanocluster with ultraviolet rays, and obtaining a fluorescence spectrum.

2. The method of claim 1, further comprising:
deriving a concentration of the cadmium on the basis of the fluorescence spectrum.

3. The method of claim 1, wherein the albumin is ovalbumin or human serum albumin or mixtures thereof.

4. The method of claim 1, wherein the reducing agent is at least any one selected from the group consisting of sodium hydroxide (NaOH), ascorbic acid, formic acid, sodium borohydride (NaBH₄), and oxalic acid.

5. The method of claim 1, wherein a concentration of the reducing agent in step I) is 0.25 M to 0.9 M.

6. The method of claim 1, wherein step II) is performed at a temperature condition of 45° C. to 60° C.

7. A composition for detecting cadmium comprising:
albumin and a reducing agent.

8. The composition of claim 7, wherein the albumin is ovalbumin or human serum albumin or mixtures thereof.

9. The composition of claim 7, wherein the reducing agent is at least any one selected from the group consisting of sodium hydroxide (NaOH), ascorbic acid, formic acid, sodium borohydride (NaBH₄), and oxalic acid.

10. The composition of claim 7, wherein the albumin is solid-state powder.

11. The composition of claim 7, wherein the reducing agent is solid-state NaOH.

12. A kit for detecting cadmium comprising:
the composition for detecting cadmium of claim 7.

13. The kit of claim 12, further comprising:
a heating means.

14. The kit of claim 12, wherein the heating means is any one selected from a blue LED (light emitting diode) element, a PN semiconductor, and combinations thereof.

15. The kit of claim 12, further comprising:
a fluorescent light source.

16. The kit of claim 15, wherein the fluorescent light source is a ultraviolet (UV) lamp.

17. The kit of claim 12, further comprising:
a fluorescence detector.

18. The kit of claim 12, further comprising:
a display part displaying fluorescence detection values.
